(19) United States
(12) Patent Application Publication
(10) Pub. No.: US 2011/0230713 A1
(43) Pub. Date: Sep. 22, 2011

(54) LAPAROSCOPE WITH ADJUSTABLE SHAFT

(75) Inventors: Ralf Kleemann, Hamburg (DE); Thomas Wosnitza, Lueneburg (DE)

(73) Assignee: OLYMPUS WINTER & IBE GMBH, Hamburg (DE)

(21) Appl. No.: 13/063,115

(22) PCT Filed: Feb. 24, 2009
(86) PCT No.: PCT/EP2009/001292

§ 371 Date: May 31, 2011

(30) Foreign Application Priority Data
Sep. 9, 2008 (DE) 10 2008 046 463.5
Sep. 9, 2008 (DE) 10 2008 046 464.3

(51) Int. Cl. A61B 1/00; A61B 1/06

(52) U.S. Cl. 600/106; 600/178

(57) ABSTRACT

A laparoscope with an elongated rigid shaft, including an observation device at its distal end and a handle body at its proximal end, with a main body disposed at a distance from the handle body and connected thereto by a connecting piece, wherein the connecting piece is designed such that the position thereof can be adjusted.

### CROSS-REFERENCE TO RELATED APPLICATION

The present application is based upon and claims the benefit of priority from the PCT/EP2009/001292 filed on Feb. 24, 2009, which claims benefit to DE 10 2008 046 463.5 filed on Sep. 9, 2008 and DE 20 2008 046 464.3 filed on Sep. 9, 2008, the entire contents of each of which are incorporated herein by reference.

### BACKGROUND

Field

The present invention generally relates to surgical endoscopes, and particularly to a laparoscope as well as a laparoscopic system.

Laparoscopes generally comprise an elongated thin shaft, with which they can be introduced into the abdominal cavity via a laparoscopic port penetrating the abdominal wall in order to observe operations with an observation device located at a distal end of the shaft there. The observation device operates with the aid of a lens through a window within the shaft. From there the image obtained must be transmitted from within the patient to the eye of the surgeon. This can be realised in a number of different ways.

An optical image transmitter extending along the entire length of the shaft can be provided, which is equipped with lenses arranged in series or with an image transmission fibre bundle. The image produced by the lens can also be recorded by an electronic image sensor and transmitted via an electric cable or in a wireless manner. Nowadays the image is usually finally transformed into a digital format and displayed on a monitor.

The main body is generally located at the end of the shaft, the former being characterised mainly by an enlarged cross-section compared with the shaft, which is possible for the main body, as the same remains outside of the body of the patient. Bulky items such as for example circuit boards, for which there would be no room in the narrow cross-section of the shaft, can therefore be located in the main body. Operating means and display means such as signal lights can also be provided on the main body.

With known laparoscopes of this kind the shaft itself is rigid and straight. In the application position of the shaft, namely substantially at an angle to the abdominal wall, its proximal end section serving for actuation of the same projects towards the outside at a corresponding angle to the abdominal wall with the main body, and is thus located within an area that is required for a number of other instruments, in particular for laparoscopic shaft instruments, the shafts of which are also located in the abdominal cavity and must be operated via their externally positioned actuating end part.

This mutual obstruction of instruments with the laparoscope occurs in particular when a port is used that can allow simultaneous access to the abdominal cavity to several shafts. The proximal actuating end part of the instruments and the laparoscope located outside of the body of the patient are then located especially closely together and mutually obstruct each other.

It is the purpose of the present invention to develop a laparoscope in such way that the mutual obstruction between the laparoscope and other laparoscopic instruments described above can be avoided.

### SUMMARY

According to the invention the rigid shaft provided for insertion into the abdominal cavity via a port ends at a handle part located proximally on the shaft. The main body is located apart from the handle body and is connected with the same via a connecting piece, whereby the connecting piece is position adjustable. The position of the main body can thus be adjusted in relation to the rigid shaft and the handle piece, so that the problems of prior art can be solved. The main body therefore no longer necessarily forms a straight extension of the rigid shaft in an area where it obstructs other instruments. Instead it can be pivoted to one side into an area where it no longer causes an obstruction. The problem of obstructing other laparoscopic instruments with the laparoscope is therefore solved.

The characteristics of claim 2 are preferably provided. The connecting piece is equipped with a bending means with which either the connecting piece as a whole or the two parts of the connecting piece adjacent to the bending means can be bent relative to each other. The bending means is located proximally to the handle piece. By adjusting the angle of the bending means located proximally to the handle body in relation to the rigid shaft positioned in the body the connecting piece with the proximally connected main body can be moved away from the area that is also required by the other instruments. The bending means is designed in such a way that it can withstand certain forces without changing its angle, in particular those that are so great that the bending means itself can hold the laparoscope in an desired angle position. The, for example, diagonally upwardly projecting main body will therefore not lower under its own weight, or for example lower under the weight of a cable extending away from the same. The bending forces are however also so great that the surgeon holding the main body can manipulate the distal end of the shaft without the angle changing. On the other hand these forces must of course be so low that the laparoscope is not destroyed upon application of the same.

In addition they must not exceed a parameter that can be easily generated, so that the surgeon can in particular realise the angle adjustment in situ, particularly also by hand. For such an angle adjustment the surgeon can for example hold the handle body and the main body and apply the necessary bending force to realise the angle adjustment.

According to claim 3 it is of advantage to design the bending means as a joint. This joint can be of short length, for example not substantially more than the diameter of the connecting piece. The adjacent parts of the connecting piece can be rigid and straight here. The joint can be designed to be pivotable around an axis that is vertical in relation to the shaft, or for example also as a ball joint bendable on all sides. The joint can also be positioned directly between the handle body and the connecting piece.

According to claim 4 the bending means can alternatively be designed as a bendable section of the connecting piece, which is bendable under the influence of the necessary forces and is for example designed to be bendable into a consistent arc. The connecting piece can be bendable area by area, or be designed to bend as a whole.

According to claim 2 the invention provides that the bending means is designed to be self-supporting. This can be realised in various ways, for example by means of steering heaviness due to friction forces, which would be of particular advantage with a construction according to claim 4, or for example with fixing means that can be loosened prior to adjusting, and will in particular be of constructional advantage when the bending means is designed as a joint. The loosening and fixing can for example be activated via a switch on the main body.

According to claim 5 the main body is preferably at least in part formed as a handle. From here the shaft can be manipulated, whereby the self-supporting characteristic of the bending means guarantees a safe, problem-free manipulation.

According to claim 6 the alternative design of the connecting piece is preferably bendable in a non-self-supporting way. This means that the bendable part cannot support the main body, so that the same will lower until it abuts, for example, against the abdomen of the patient or on the operating table. This also means that this bendable part will not bounce back following bending and will be so floppy that the free manipulation of the rigid distal shaft is not substantially obstructed whilst the main body remains fixed. The bendable connecting piece can for example have the bending characteristics of an electric cable or a soft hose. It guarantees free space beyond the proximal end of the rigid shaft or beyond the adjacent handle part, so that the external parts of other instruments can be moved and operated freely there.

The fact that the connecting piece is not self-supporting also means that it cannot transfer actuating power or manipulation power to the rigid shaft. In order to manipulate the same the rigid shaft itself must therefore be held, namely at its proximal end. The handle piece is thus preferably located there to ensure that the same can be held easily. This handle piece can in some cases also be designed as a holding coupling or a coupling of the type with which the laparoscope can for example be coupled with a tripod or another holding means.

Even when the connecting piece is only very short and permits only a bending of the main body in a downward direction this alone creates more room above the abdomen of the patient in the sense of the task to be solved herein. However, according to claim 7 the bendable extension piece is preferably of sufficient length to allow a positioning of the main body on the abdomen of the patient, or even on the operating table. For this the length should suffice to be able to lie the connecting piece down in a strain-relieved way to prevent obstruction of the rigid distal shaft during manipulation.

The characteristics of claim 8 are preferably provided. In this way the laparoscope is designed as a video laparoscope and enjoys the advantages of this construction principle that also facilitate the design of the invention, as the electric lines will for example not obstruct the bending means.

The image sensor according to claim 9 is preferably rotatably located in the shaft. This is particularly important with laparoscopes where the viewing direction of the image sensor is diagonal in relation to the axis of the shaft. By rotating the image sensor the physician can maintain the image in an upright position on the observation monitor at all times. An activating member for the rotation adjustment of the image sensor is located on the handle body here, namely where the same would have to be held in any case, in order to direct the rigid shaft inside the body. In a simple design the handle body can for example be formed as a thickening of the shaft end.

According to claim 10 the activating member is preferably formed as a rotatable ring that enables a simple intuitive operation.

The illumination of the operating area can be realised with the aid of an illumination means that is completely separate from the laparoscope. The characteristics of claim 11 are however preferably provided. In this way the laparoscope itself can be used for illumination. The easily bendable optical fibre bundle will not obstruct the adjustment of the connecting piece.

According to claim 12 the main body preferably comprises electric and/or electronic components such as for example circuit boards, amplifiers and such-like, as there is more room here than in the narrow shaft.

According to claim 13 switches with which the physician controlling the laparoscope can control various functions, such as for example functions of an image recording camera or recording means, light functions such as for example brightness, light tone or other functions connected with an operation, such as for example the height control of the operating table, are preferably located on the main body.

The characteristics of claim 14 are preferably provided. A cable of this type can accomplish the supplying of the illumination light as well as the transfer of images via electric lines or, for example, via a flexible image conductor, if the image is not transmitted wirelessly via a radio link. The introduction of illumination light and the transfer of the images can however also be realised via separate cables, although a joint cable is also possible.

According to claim 15 the connecting piece is particularly preferably designed in the form of a goose neck that can be produced to provide varying bending stiffnesses and any length in a relatively easy way. In addition goose necks have the advantage that the interior comprises a free lumen. It is therefore possible, and preferable according to claim 16, to route the electric lines along the lumen enclosed by the goose neck.

One could also arrange the optical fibres in this lumen enclosed by the goose neck. However, according to claim 17 it is suggested to be of advantage if the optical fibres can be located in an annular space surrounding the goose neck, the same extending between the outer surface of the goose neck and a radially distant outer protective sleeve. The goose neck can then be designed with a smaller diameter and a lower weight can thus be achieved for the connecting piece.

According to claim 18 the goose neck is preferably radially enclosed by an inner protective sleeve to prevent damage through friction on the goose neck to the optical fibres. For this the optical fibres are located between this inner protective sleeve and the outer protective sleeve.

Preferred is a laparoscopic system according to claim 19, which preferably also provides a surgical shaft instrument in addition to the laparoscope in line with claim 20.

The drawings schematically illustrate the invention by way of an example and show the following in a simplified and schematic form:

### BRIEF DESCRIPTION OF THE DRAWINGS

FIG. 1 shows a side view of a laparoscope according to a first embodiment;

FIG. 2 shows a side view of a laparoscope according to a second embodiment;

FIG. 3 shows a side view of a laparoscope in a third embodiment; and

FIG. 4 shows a longitudinal section through the bendable connecting piece of FIG. 2.

### DETAILED DESCRIPTION

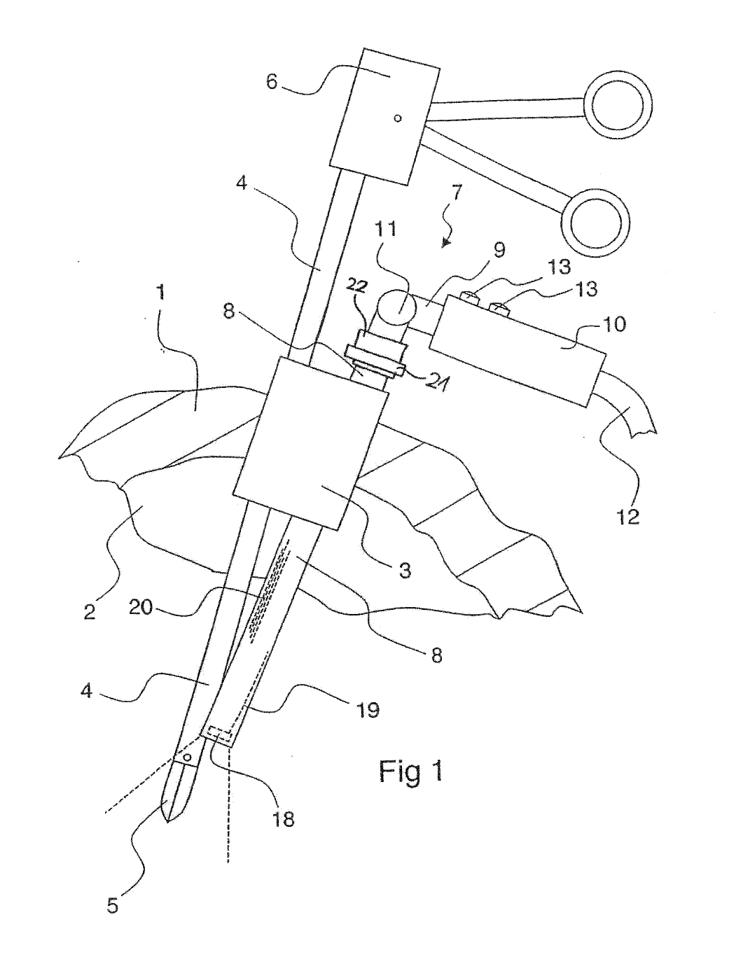

FIG. 1 shows a section of an abdominal wall 1 of a patient, under which the abdominal cavity 2 has been inflated to create room to allow free working with instruments. A port 3 extends through the abdominal wall to enable sealed access to the abdominal cavity 2 from the outside.

The port 3 consists of a multiple port that allows the simultaneous introduction of several shafts. The port 3 is inserted through an incision opening in the abdominal wall 1 with its highly schematically illustrated outer housing. The port 3 thus creates a passage that extends from the outside through the port 3 into the abdominal cavity 2. This passage is sealed with suitable means not shown here to be able to maintain the gas overpressure within the abdominal cavity 2. These sealing means are designed in such a way that shafts of instruments can be introduced through the same.

In the example illustrated in FIG. 1 laparoscopic forceps with a forceps shaft 4, at the distal end of which the illustrated jaws 5 of the forceps and at the proximal end of which an activating means 6 with the two finger grips shown are located, have been inserted through the port 3, whereby the same are moveable against each other for opening and closing the jaws 5 of the forceps.

A laparoscope 7 consisting of a rigid distal shaft 8, a proximal adjacent handle piece 22 and a rigid proximal connecting piece 9 is further introduced through the port 3. At the proximal end of the connecting piece 9 a main body 10 is located.

The connecting piece 9 is divided into two parts, connected with each other via a joint 11 that in this embodiment is angle adjustable in relation to an axis vertical to the drawing plane and enables the adjustment of the proximal part of the connecting piece 9 into the substantially right angled bent position of FIG. 1 in the same way as into a straight extended form, in which the connecting piece 9 is in a straight extended form, in which the connecting piece 9 stands as a straight extension of the shaft 8, or into various other angle positions. The joint 11 can for example also be designed as a ball joint to enable angle adjustments on all sides.

The joint 11 should be designed self-supporting, e.g. should provide holding forces, that will suffice in all instances to maintain the angle position set to withstand the usual forces applied during a laparoscopic procedure. In particular it can be held at the main body 10 in order to manipulate the shaft 8 in the usual way necessary for an operation.

The holding force at the joint 11 can for example be generated by way of friction forces or through detachable brakes that can for example be activated from the main body 10. The joint can for example also be equipped with catches, for example at 5° intervals.

An observation device 18 illustrated with the aid of a broken line is provided in the distal end area of the rigid shaft 8 of the laparoscope 7, which enables a view through a window in the distal end of the shaft 8 towards the outside. Conventional construction types used for endoscopes can be utilised here. A lens that generates an image that can be transmitted in a suitable way is provided for this. Inside the shaft 8 and the connecting piece 9 the image can be transported by way of a relay lens arrangement. The image can for example be transported via a flexible optical fibre bundle through to the joint 11. An electronic image sensor 18 can also be provided in the distal end area of the shaft 8, which will be of advantage for the laparoscope 7 of the construction type illustrated here, as the image is transported from the image sensor 18 via electric lines 19 that are easy to route through the joint 11.

The necessary illumination of the dark abdominal cavity 2 can be realised by other means, for example with a separately introduced illumination means. With the construction type that is usual for endoscopes the illumination can however be realised through the shaft 8 and the connecting piece 9, namely by means of the conventional optical fibre bundle 20 routed through the same, which provides light radiation on the distal facing surface of the rigid shaft 8 in the area of the opening angle illustrated.

The electric lines, optical fibres and suchlike running through the shaft 8 and the connecting piece 9 extend up to the main body 10 and can be routed through the same into a further cable 12 up to a connected apparatus not shown here, which provides light, and processes and displays the video signals. The transmission from the image sensor 18 in the distal tip area of the shaft 8 to such a processing apparatus can also be realised in a wireless manner via a suitable radio link.

Key switches 13 are located on the main body 10, where they are easy to activate in the direct working vicinity of the surgeon without the same having to take his attention away from the location of the operation. These switches 13 can be connected via lines to a remotely located control means or can be wireless, and can control various functions that may be required during the operation. In this way it is for example possible to record and store images, and to adjust the brightness. It is possible to realise picture rotations, or to activate completely different functions such as for example a height adjustment of the operating table.

According to known prior art the shaft 8 extends to the main body 10. The main body would project as an extension of the shaft 8 in a proximal direction, e.g. into the area that is required as a working area for the actuating means 6 of the forceps 4, 5, 6. This mutual instrument obstruction is rectified by the present invention.

The obstructing main body 10 can be pivoted out of the area of the forceps actuating means 6 with the aid of the joint 11, for example into the angled position of FIG. 1, in which the main body 10 does not obstruct the forceps actuating means 6.

FIG. 1 further shows a handle piece 22 on which a ring 21 is rotatably located. The rotatable ring 21 controls the image sensor 18 rotatably positioned inside the shaft 8 via internal adjustment connections, for example via magnetic couplings or electric motor adjustment connections. The surgeon, who has one hand on the handle piece 22 anyway, can easily activate the rotating ring 21 there.

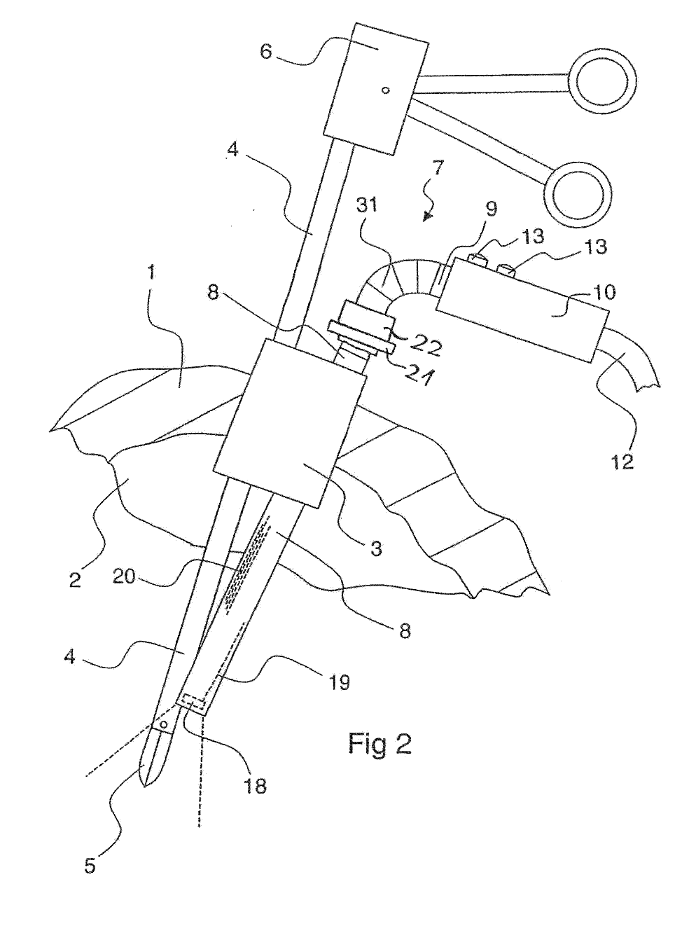

FIG. 2 shows a second embodiment of the laparoscope 7 of the invention, where identical reference numbers to those in FIG. 1 have been used wherever possible.

The application location, the port 3 and the forceps 4, 5, 6 shown are identical to those illustrated for the embodiment of FIG. 1.

The laparoscope 7 agrees with all parts of those of FIG. 1 with the exception of the joint 11 illustrated there, which has been replaced with a bendable connecting piece 31 for the embodiment of FIG. 2, the same being located between the handle piece 22 and the main body 10, which are in turn identical to those of the embodiment of FIG. 1, and the which enables the same angle adjustment as the one shown in FIG. 1. Once again all angle positions can be set here. The connecting piece 9 can be completely bendable, or only in certain areas.

The bendable connecting piece 31 is also self-supporting, e.g. it is equipped with sufficient steering heaviness as one knows from, for example, bendable rigid hoses, which are for example known by the name “goose neck” in prior art.

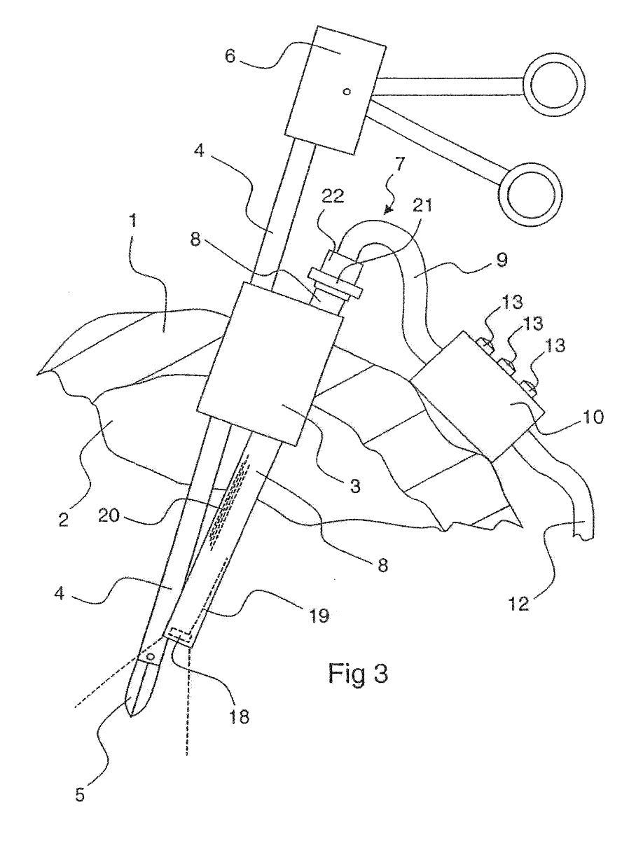

FIG. 3 shows a third embodiment of the laparoscope 7 of the invention, where the same reference numbers as those used for FIGS. 1 and 2 are used wherever possible.

The connecting piece 9 proximally adjacent to the handle body 22 is not self-supporting bendable in this embodiment, e.g. it is so floppy that the main body 10, if one lets go of the same, will immediately drop down onto the abdominal wall 1. The connecting piece is not self-supporting and cannot hold the main body 10 upright. This also means that the shaft 8 can be manipulated freely without being affected by the main body 10 positioned at a distance.

The obstructing main body 10 can be bent away from the area of the forceps actuating means 6 with the aid of the bendable connecting piece 9 and deposited in a downward position. In this embodiment the main body 10 lies on the abdomen of the patient, as shown in FIG. 3. If the bendable connecting piece 9 is longer the main body 10 can for example also be deposited on the table next to the patient.

A handle piece 22 that can be more easily held in one hand than the very thin shaft 8 itself is located at the proximal end of the rigid shaft 8. The position of the rigid shaft 8 can thus be manipulated in the desired way, for example to direct the area of view in front of the distal end of the shaft 8 onto the jaws 5 of the forceps indicated by means of the broken lines, as illustrated in FIG. 3.

The bendable connecting piece 9 should be at least so long that the hand will not come into obstructing contact with the main body 10 when the handle piece 22 is held in one hand in order to manipulate the shaft 8.

A holding coupling not shown here, with which the rigid shaft 8 can for example be coupled to and held with, for example, a tripod in order to free the hands of the surgeon, can be provided instead of the handle piece 22. The handle piece 22 itself can serve as a holding coupling for a suitable tripod positioning.

FIG. 3 shows a ring 21 on the handle piece 22, which is rotatably located there. The rotatable ring 21 controls the image sensor 18 rotatably located in the rigid part 8 of the shaft via internal adjustment connections, for example via magnetic couplings or electric motor adjustment couplings. The surgeon, who already has one hand on the handle piece 22, can easily actuate the rotatable ring 21 there. Instead of a rotatable ring a differently designed actuating member, for example a rotating lever or suchlike, can also be provided.

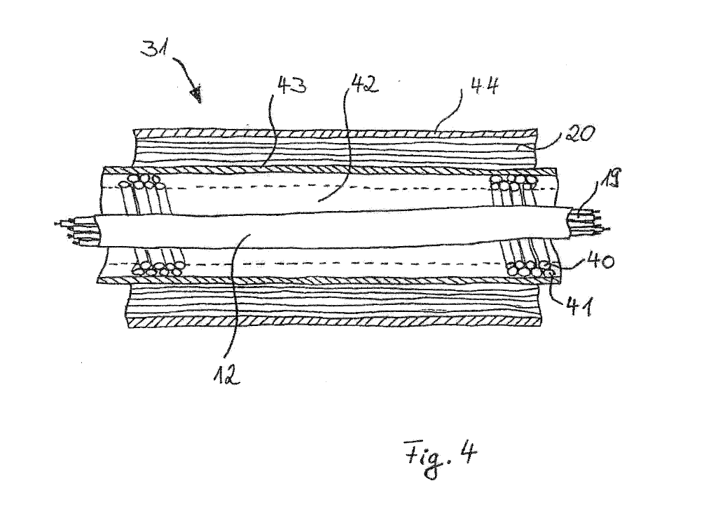

FIG. 4 shows a longitudinal section through the bendable connecting piece 31 of FIG. 2 which is formed as a goose neck consisting of two coil springs 40, 41 wound offset, which enclose a free lumen 42 within their core. An inner protective sleeve 43 encloses the goose neck against the outside. Optical fibres 20 for transmitting illumination light to the distal end of the shaft 8 are routed in an annular space delimited against the inside by said protective sleeve 43, and against the outside by a radially distantly positioned outer protective sleeve 44. A cable 12 with electric lines 19 for transmitting camera signals extends through the free lumen 42. The inner protective sleeve can also be omitted. It does however offer the advantage that the placing of a load on the optical fibres through friction on the goose neck will thus be avoided, in particular when the goose neck is bent.

1. A laparoscope with an elongated rigid shaft, comprising an observation device at its distal end and a handle body at its proximal end, with a main body disposed at a distance from the handle body and connected thereto by a connecting piece, wherein the connecting piece is designed such that the position thereof can be adjusted.

2. The laparoscope according to claim 1, wherein the connecting piece comprises a bending means that allows a reversible angle adjustment of the connecting piece or the parts of the connecting piece adjacent to the bending means in relation to each other when actuating forces are applied that will not destroy, but that are greater than the self-supporting force as well as the forces present at the bending means if forces for the manipulation of the laparoscope are applied.

3. The laparoscope according to claim 2, wherein the bending means is designed as a joint.

4. The laparoscope according to claim 2, wherein the bending means is designed as a connecting piece that is bendable at least in some areas.

5. The laparoscope according to claim 2, wherein the main body is formed as a handle at least in some areas.

6. The laparoscope according to claim 1, wherein the connecting piece is designed not to be self-supportingly bendable.

7. The laparoscope according to claim 6, wherein the connecting piece is of a length that allows the depositing of the main body next to the port in the application position of the laparoscope in a laparoscopic port.

8. The laparoscope according to claim 1, wherein the observation device is designed as an image sensor located at the distal end of the shaft, the same being connected by means of continuous electric lines via the shaft and the connecting piece.

9. The laparoscope according to claim 8, wherein the image sensor is designed rotatably and is in adjustment connection with an actuating member located on the handle body and actuatable from the outside.

10. The laparoscope according to claim 9, wherein the actuating member is designed as a ring rotatably positioned on the handle piece and being rotatable around the axis of the handle piece.

11. The laparoscope according to claim 1, further comprising an optical fibre bundle serving for illumination, the optical fibre bundle being routed through the shaft and the connecting piece.

12. The laparoscope according to claim 1, wherein the main body comprises electric and/or electronic components.

13. The laparoscope according to claim 12, further comprising at least one switch to be activated from the outside, the at least one switch being located on the main body.

14. The laparoscope according to claim 8, further comprising a cable containing an electric and/or optical fibre line leading away from the main body.

15. The laparoscope according to claim 4, wherein the connecting piece is designed as a goose neck.

16. The laparoscope according to claim 15, wherein the observation device is designed as an image sensor located at the distal end of the shaft, the same being connected by means of continuous electric lines via the shaft and the connecting piece and the electric lines are routed through the lumen enclosed by the goose neck.

17. The laparoscope according to claim 15, further comprising an optical fibre bundle serving for illumination, the optical fibre bundle being routed through the shaft and the connecting piece wherein the optical fibres are located within an annular space surrounding the goose neck, the same being formed between the outer surface of the goose neck and a radially distanced outer protective sleeve.

18. The laparoscope according to claim 17, wherein the goose neck is radially surrounded by an inner protective sleeve, and in that the optical fibres are located between this inner protective sleeve and the outer protective sleeve.

19. A laparoscope system with a port designed for the simultaneous passage of several shafts and with a laparoscope according to claim 1.

20. The laparoscope system according to claim 19, wherein a surgical shaft instrument is provided.
